(19) United States
(12) Patent Application Publication
(10) Pub. No.: US 2006/0018525 A1
(43) Pub. Date: Jan. 26, 2006

(54) Method and system for temporal spectral imaging

(76) Inventor: Randall L. Barbour, Glen Head, NY (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/186,568

(22) Filed: Jul. 21, 2005

Related U.S. Application Data
(60) Provisional application No. 60/589,837, filed on Jul. 21, 2004.

(51) Int. Cl. G06K 9/00; A61B 5/00

(52) U.S. Cl. 382/128; 600/319

(57) ABSTRACT

A method for imaging functional states of tissue associated with provocation dependent tissue-vascular responses is described. In its preferred embodiment the method combines dynamic near infrared optical tomographic imaging with feature extraction methods to produce a composite image whose particulars can serve to differentiate healthy tissue from diseased, monitor tissue response to therapy or measure tissue responses to pharmaceutical agents. When combined with simultaneous multisite optical measurements, the described method can be used to differentiate local and system-wide responses.

### FIELD OF INVENTION

The invention relates generally to imaging tissue function and, more particularly, to the use of near infrared imaging methods for characterizing tissue-vascular interactions for the purpose of disease detection, monitoring response to therapy and actions of pharmaceuticals.

### DESCRIPTION OF RELATED ART

The noninvasive assessment of functional states of tissue has long been recognized as a desirable approach to understanding and evaluating the health status of tissue.

Approaches used vary from routine techniques, such as an electrocardiogram, to use of complex imaging systems that are sensitive to features other than the structural components of tissue (e.g., functional magnetic resonance imaging (fMRI), magnetoencephalography MEG)). Imaging methods offer the advantage of spatially localizing particular contrast features non-invasively. Unlike anatomical imaging methods, whose contrast maps are based on elements of the anatomy, contrast maps associated with functional imaging techniques often reveal complex interactions among the different structural elements. For instance, the technique of magnetoencephalography (MEG) is based on detection of the minute magnetic fields associated with neural activity of the brain. Functional magnetic resonance imaging (fMRI) is sensitive to the changes in deoxyhemoglobin, whose level is closely linked to tissue metabolic demand. Information regarding functional states can also be revealed using radioisotopic imaging techniques such as positron emission tomography (PET) and single photon emission computed tomography (SPECT). In these cases, the resultant contrast maps identify the level of particular molecular species that may be involved in certain metabolic pathways or associated with specific disease processes.

As with any diagnostic/monitoring tool, the information value of imaging methods depends on its inherent discriminatory capability in differentiating healthy tissue from diseased, usually expressed in terms of diagnostic sensitivity and specificity, and by a host of practical issues including, cost, system size, ease of use, risk, limitations etc. In the case of anatomical imaging methods, utility is typically closely tide to the spatial resolution and its soft tissue contrast. Distortions in anatomical features or changes in contrast can reveal the presence of disease. It is generally regarded that such changes only occur much later in disease processes. The first events are those that impact on tissue function.

Another factor influencing the information value of imaging techniques is the range of contrast features that can be explored. For instance, MEG is almost exclusively limited to brain studies as it is only this organ that has such an extensive concentration of neural activity. Still other factors that impact on the utility of imaging methods, are particular features of the measurement process itself. Key among these is the temporal resolution of the technique. Thus, for instance, PET and SPECT methods are poorly suited to explore dynamic events as these often require several minutes of signal acquisition to form an image. In other cases, the information value is restricted by constraints imposed by system design that limit the conditions under which imaging studies can be performed. Most often subjects are studied while lying motionless. In many cases, especially for functional studies, this need severely restricts the utility of the technique.

It follows from these considerations that it would be especially useful to have available a functional imaging tool that can be applied to examine a range of tissue types, is sensitive to a broad range of functional states and can be employed under a broad range of measurement conditions.

Guiding consideration of this problem is the idea that among the many functional processes of the body, one especially critical to normal tissue function and health is the tight coupling between tissue metabolic demand and its vascular supply. This coupling is known to occur on a local and system-wide level. For instance, locally increased blood flow to a muscle can occur upon exercise without significantly impacting blood distribution elsewhere. In other instances system-wide effects can occur, for example, in response to shock or exposure to extreme temperatures. The breadth of functional responses of tissue that can produce a change in its blood supply is very large and has both local and system-wide origins. In addition, these responses can be additionally modified by a host of internal and external effectors. It would be particularly useful to have available a general purpose device that is capable of defining these varied states and their response to stimuli on a local and system wide level. It would also be useful to implement this in ways that provide for economical, scalable, and portable systems that can be easily configured to accommodate a range of clinical environments.

### SUMMARY OF THE INVENTION

The present invention provides the above and other advantages by providing a technique for imaging tissue in a human or other animal that takes advantage of the fact that tissues differ in terms of their vascular density and reactivity to a provocation, whether caused by internal or external stimuli. By applying a specific provocation to a patient and dynamically imaging one or more sites in the subject, multiple contrast features of the tissue can be ascertained.

In a particular aspect of the invention, a method for imaging living tissue in a subject or animal includes performing dynamic optical tomographic imaging of at least one site that comprises tissue having different vascular densities to obtain at least one time-series of data, and using this to ascertain different functional features of the tissue that are associated with the different vascular densities and associated reactivities.

### DETAILED DESCRIPTION

Biological systems can be viewed as having spatially and temporally varying properties whose function is maintained through actions occurring both locally and system wide. Specifically, it is known that integrated body function, and its regulation, is controlled through the demands of local tissues and their connectivity established by the peripheral vascular and nervous systems. Of particular interest is the role played by the vascular system or vasculature, including veins, arteries, and microvessels. Among its many functions, the vasculature serves as a conduit to provide essential nutrients to tissues and gas exchange, chief of which is the delivery of oxygen which occurs through its binding to hemoglobin. The vascular system also has a number of other properties that, in combination with these, provide a guide to develop a general-purpose tool having the suggested capabilities.

Properties of particular significance are the finding that vessel density, and its reactivity to internal and external stimuli, varies greatly among the different tissue types. This observation leads to the consideration that the particular response characteristics of the vasculature in any given tissue will vary in accordance with the type and details of the provocation. Notably, the form of this response falls into two distinct categories. Provocations can result in a change in tissue metabolic demand and in turn caused an increase in blood flow. In cases where the resulting vascular response is insufficient to meet the demand, changes in hemoglobin oxygenation will occur causing an increase in deoxyhemoglobin levels. The other response can involve changes in vascular reactivity. This is a broad class of responses that serve to adjust blood flow to tissue on a local and system-wide basis. One example, well studied, is the occurrence of vasomotion that is mainly associated with the microvasculature. In many instances, both types of response occur concurrent to a provocation.

We believe that characterization of these responses can provide a wealth of discriminatory features that can serve to distinguish one tissue type from another, healthy tissue from disease and explore actions of pharmaceutical agents, among other capabilities. While the above considerations are generally appreciated, what is not obvious is just how objective measurement of the various responses can be achieved in ways that lend themselves to specific tissue characterization.

The close linkage between tissue function and its vascular supply, in particular, the availability of oxygen, strongly suggest that measures of hemoglobin states in bulk tissue structures, at rest or in response to stimuli, could provide a basis for developing the suggested capability. An elementary consideration, though important, is the fact that in addition to being the principal vehicle for oxygen delivery to tissue, hemoglobin is normally confined to the vascular space. Yet another consideration is the fact that different elements of the vascular tree have distinct natural beat frequencies. It follows from this that measures of the time variations in hemoglobin states in tissue provide a direct measure of vascular reactivity while simultaneously revealing changes in metabolic demand. A key element of the current invention is the recognition that this dual property combined with the known variations in vascular density and reactivity to provocation among the different tissue types provides a basis for specific tissue characterization. On a physical level, the elementary basis of this characterization follows from the fact that different tissues have different temporal and spectral properties owing to their vascular content and intrinsic responsivity to stimuli.

The range of provocations that could be considered is large, varying from simple manipulations to complex. Generally, the provocation can be selected to cause a change in one or more of the different types of the vasculature. Moreover, the patient can be subject to the provocation before or during an imaging study. Still further, multiple provocations can be applied, and/or directed to one or more sites of the patient. Different types of provocations can be catalogued so that a particular provocation can be selected that causes a particular change in the vasculature of tissue in a region of interest in a patient. In some instances, natural background fluctuations in the vasculature may be sufficient to allow for tissue discrimination.

As noted, another property of the vasculature having significance is the well-known observation that the natural temporal dynamics of the vascular tree differ considerably among its principal elements. For instance, the arterial tree exhibits a dominant cardiac frequency. Likewise, the venous tree has a natural respiratory beat frequency. Different still are the temporal properties of the microvessels, which respond to neural, hormonal, and local metabolic controls. This suggests that use of particular classes of numerical methods e.g., signal separation techniques, could allow for improved characterization of the vascular response.

Yet another property of the vasculature that serves to guide development of a practical system is the finding that among the naturally occurring chromophores, hemoglobin is the only one that has a dominant temporal signature. This occurs because, as noted above, hemoglobin is ordinarily confined to the vascular space which exhibits a range of natural beat frequencies.

Still another property of the vasculature and its interaction with tissue that can be exploited to differentiate healthy tissue from diseased is its connectivity. One consequence of this is the ability to shunt blood from one region of tissue to another, or in cases of extreme stimuli (e.g., shock), from one area of the body to another. This observation leads to the suggestion that assessment of vascular dynamics at multiple sites simultaneously could provide for improved understanding of integrated body function and synchrony, among other capabilities. It could even allow for more sensitive detection of early disease states. For instance, in the case of breast cancer, a normally unilateral disorder having an aberrant vascular network, the focal detection of desynchronized responses to provocation might suggest the presence of a tumor.

While a number of techniques could be considered to explore one or more of the above features, best suited are optical methods, especially near infrared techniques. At these wavelengths, where deep penetration occurs, it is possible to discriminate between changes in tissue blood volume and oxygenation and to explore the temporal dynamics of the hemoglobin signal. Practical approaches are available that allow for the capture of a time-series of optical tomographic measurements from which can be computed a corresponding time-series of 3D images. See PCT publication WO 01/20305, published Mar. 22, 2001, entitled Method And System For Imaging The Dynamics Of A Scattering Medium, incorporated herein by reference. We have also described use of numerical methods that minimize the image degrading effects caused by the expected uncertainties of experiment (see PCT publication 03/012,736, published Feb. 13, 2003, entitled Method And System For Enhancing Solutions To A System Of Linear Equations, incorporated herein by reference), techniques to improve image quality (see PCT publication 01/20307, published Mar. 22, 2001, entitled Method And System For Enhanced Imaging Of A Scattering Medium, incorporated herein by reference), and methods for isolating and quantifying the dynamics of complex overlapping signals (see PCT publication 03/063366, published Jul. 31, 2003, entitled Normalized-Constraint Algorithm For Minimizing Inter-Parameter Crosstalk In Imaging Of Scattering Media, incorporated herein by reference), the type of which are certainly represented by the architecture of the vasculature tree.

Knowledge of these features and capabilities leads to the realization that significantly improved insight into tissue function and detection of disease states could be gained by extending the method of dynamic optical tomography in ways that provide for the detection of multiple contrast features associated with one or more provocations. In practice this could be accomplished by analyzing time-series diffuse optical image data using methods that are sensitive to the different state functions of the tissue. For instance, the natural differences in vascular compliance associated with the different elements of the vascular tree could be detected by determining the rate of increase in local tissue blood volume caused, for instance, by inflation of a pneumatic cuff to cause mild venous occlusion. Large veins would be expected to expand most easily distal to the site of occlusion, followed a rapid return to baseline in response to resumption of normal flow. The major arteries, on the other hand, having thick muscular walls, should distend less rapidly. Regions of tissue dense with microvessels (e.g., muscle), might exhibit a prominent hyperemic overshoot upon release of the indicated provocation as a consequence of vascular congestion. In fact, it has been our experience that those regions that exhibit the expected response exhibited by a vein, fail to exhibit the hyperemic overshoot that is seen in regions containing muscle, thus demonstrating structure-specific functional behavior. This observation is entirely consistent with the fact that tissues vary greatly in their vessel density and response to internal and external stimuli. Thus, even with a provocation as simple as induced mild venous occlusion, markedly different responses are observable. Different forms of provocations will therefore produce different responses in accordance with different tissue types and the associated vasculature. The details of these responses can serve as discriminators of disease, actions of pharmaceutical agents and general insight to normal tissue function.

A similar analysis could be applied to discern variations in other state functions, such as peripheral vascular resistance. This could be accomplished, for example, by comparing the response before and after a local provocation to tissue (e.g., mild heating of peripheral muscle followed mild venous occlusion). To summarize, a key idea here is that because tissues vary greatly in their vessel density and response to internal and external stimuli, multiple contrast features can be identified for any given tissue structure.

It also follows that additional discriminatory information could be gained by monitoring more than one tissue site simultaneously. This information could be further augmented still by the simultaneous capture of other time-series measures (e.g., ECG, EEG, pulse oximetry, arterial tonometry, EMG) whose signals, or features thereof, could be used to discriminate tissue responses, deconvolve local responses from overlapping global signals among other uses. Still further information could be gained by combining imaging information obtained from dynamic optical tomography (DOT) measures using strategies suggested here with anatomical maps generated using structural imaging methods.

As described by the inventors in PCT publication 01/20306, published Mar. 22, 2001, entitled System And Method For Tomographic Imaging Of Dynamic Properties Of A Scattering Medium, incorporated herein by reference, one approach is to implement a collection of the optical data using a time-multiplexed source together with parallel detection and use of gain switching techniques. Inversion of this information to yield an image that is robust to the expected uncertainties of experiment and computationally efficient is ordinarily a difficult task. One approach that has proven especially effective is a technique known as the Normalized Difference Method (NMD). See U.S. patent application publication no. 2004/0010397, published Jan. 15, 2004, entitled Modification Of The Normalized Difference Method For Real-Time Optical Tomography, and incorporated herein by reference. This approach has proven very effective in discerning the temporal properties of highly scattering media, but yields images having reduced spatial resolution. A robust and efficient spatial deconvolution scheme that can significantly improve on the image quality achieved using the NDM approach is described in PCT Publication WO 2005/006962, published Jul. 27, 2005, entitled Image Enhancement By Spatial Linear Deconvolution, and incorporated herein by reference. Use of these techniques and data collection methods leads to the generation of a time-series of images that can be subjected to any of a number of signal processing methods to allow for the extraction of one or more feature maps. An overlay of this information can provide a database that serves to characterize the response(s) features of tissue as a function of different provocations. The resulting information, which makes use of both temporal and spectral properties of tissue, can subsequently serve in the suggested ways. It is also appreciated that whereas these properties may be derived from analysis of the hemoglobin signal (comprising measures of oxyhemoglobin, deoxyhemoglobin, total hemoglobin or hemoglobin oxygen saturation), there are other naturally occurring chromophores observable at near infrared wavelengths that could also be explored. For instance, it is appreciated that tissue water and lipid content can be assess as can be the wavelength dependent scattering properties as defined by measures of scattering power and scattering amplitude. It is further appreciated that other types of optical signals may be endogenously present as a result of use of genetic engineering techniques that serve to produce luminescent or fluorescent chromophores.

Whereas the above considerations have been limited to examination of naturally occurring contrast features, it is appreciated that the described technique can be additionally augmented through use of injectable contrast agents that serve to alter the optical field in tissue. This can take the form of absorbing dye such as indocynanine green and related derivatives, or use of luminescent or fluorescent dyes. In the case of the latter, the optical measurement device employed would be appropriately modified to provide for selected detection of these signals.

Having implemented the above considerations, it is appreciated that what follows is a set of image features that serve to define the response of different tissue types to one or more provocations. These features can be combined in ways that serve to produce a multi-dimensional mosaic of information. In the limit, a comprehensive library of such mosaics could be derived that serve to define tissue responses to a broad range of stimuli and experimental conditions. Having generated such a database, any of a number of multivariate statistical methods and related numerical techniques (neural network methods) could be employed that would allow, for example, to distinguish one disease state from another. In one embodiment, the technique of logistic regression could be employed wherein site/tissue-specific, independent parameters would serve to generate a composite discriminator function.

To those skilled in the art of tissue optical measurements, it will be evident that any of a number of illumination-detection strategies can be employed to provide for the above information. For instance, optical data can be collected using DC, frequency domain or time-resolved illumination-detection techniques. These methods could employ one or more illuminating wavelengths and can be implemented with or without wavelength selective filters. It also evident to those skilled in tomographic imaging methods, that any of a number of inversion schemes could be adopted to form a tomographic image. It is further understood by those skilled in the art of signal processing, that given a time-series of information, any of a number of numerical methods can be adopted to identify features of interest. Examples include, but are not limited to analysis in the time, frequency or time-frequency domains, use of time-correlation methods, and use of signal decomposition methods such a blind source separation techniques. It is further understood by those skilled in the art of multivariate analysis techniques that any of a number of regression type methods could be adopted that serve to define multivariate discriminators that can distinguish healthy subjects from those with disease, or one disease type from another. It is also understood that such analysis tools can be used in various combinations to allow for isolation of local phenomenology from system-wide responses, and for the description of functionally linked coordinated states.

The techniques of the invention can be implemented by a general-purpose computer having a memory or other program storage device for storing software, and a processor for executing the software to achieve the functionality described herein. The computer interfaces with an imaging system such as the system described in the above-mentioned U.S. patent application having docket no. 16855 to obtain a time-series of data from which the desired information can be ascertained.

The invention has been described herein with reference to particular exemplary embodiments. Certain alterations and modifications may be apparent to those skilled in the art, without departing from the scope of the invention. The exemplary embodiments are meant to be illustrative, not limiting of the scope of the invention, which is defined by the appended claims.

What is claimed is:

1. A method for imaging tissue in a living subject, comprising:
performing a time series of near-infrared optical measurement using at least one source, at least one detector, and at least one illuminating wavelength to collect time-series optical data; and using the collected time-series optical data to ascertain different features of the tissue that are associated with different types of tissue-vasculature responses.

2. The method of claim 1, wherein the time-series optical measurement is performed on the subject at rest or the living subject undergoing at least one provocation.

3. The method of claim 1, wherein the time-series optical measurement is obtained using at least one of a plurality of illumination-detection strategies.

4. The method of claim 1, wherein the collected time-series of optical data is combined to produce two-dimensional or three-dimensional maps of tissue-vascular responses.

5. The method of claim 4, wherein the two-dimensional and three-dimensional maps comprise one of a single image and a time-series of images.

6. The method of claim 1, wherein the collected time-series of optical data is processed using tomographic imaging principles to produce a spatially resolved 2D or 3D image.

7. The method of claim 1, wherein the collected time-series of optical data is processed using tomographic imaging principles to produce a spatially resolved image time-series.

8. The method of claim 7, wherein the one of a single image and a time-series of images comprise an image of oxyhemoglobin, deoxyhemoglobin, total hemoglobin, hemoglobin oxygen saturation, tissue water, lipid content, scattering amplitude, or scattering power.

9. The method of claim 1, wherein the collected time-series of optical data is processed using at least one signal processing technique to extract features that quantify the response of tissue to a provocation.

10. The method of claim 5, wherein one of the image and time-series of images is processed using at least one signal processing technique to extract at least one feature that quantifies the response of tissue to a provocation.

11. The method of claim 10, wherein the at least one feature is used to yield multi-dimensional information to further characterize tissue response to provocation.

12. The method of claim 11, wherein the multi-dimensional information is collected simultaneously from a plurality of sites on the living subject.

13. The method of claim 11, wherein the multi-dimensional information is further combined with other concurrent physiological measurements and additionally processed using signal decomposition methods that isolate local tissue-specific responses from system-wide responses.

14. The method of claim 10, wherein the multi-dimensional information is used to detect disease states, monitor tissue response to therapy, or measure tissue response to actions of pharmaceutical agents.

15. The method of claim 1, wherein the collected time-series of optical data is based on measurement of luminescent or fluorescent signals having endogenous origin.

16. The method of claim 1, wherein the collected time-series of optical data is based on measurement of luminescent or fluorescent signals that have been injected into the living subject.
